(19) United States
(12) Patent Application Publication
(10) Pub. No.: US 2004/0223918 A1
(43) Pub. Date: Nov. 11, 2004

(54) Aerosolization of cromolyn sodium using a capillary aerosol generator

(75) Inventors: Stephen Pham, Chesterfield, VA (US); Tung T. Nguyen, Midlothian, VA (US)

Correspondence Address: BURNS DOANE SWECKER & MATHIS L L P, POST OFFICE BOX 1404, ALEXANDRIA, VA, 22313-1404, US

(73) Assignee: Chrysalis Technologies Incorporated

(21) Appl. No.: 10/830,463

(22) Filed: Apr. 23, 2004

Related U.S. Application Data
(60) Provisional application No. 60/468,266, filed on May 7, 2003.

(51) Int. Cl. A61K 31/353; A61L 9/04

(52) U.S. Cl. 424/45; 514/456

(57) ABSTRACT

Liquid aerosol formulations for generating cromolyn sodium aerosols include at least one high volatility carrier and an optional additive such as a surfactant and/or low volatility liquid. In some embodiments, the liquid aerosol formulation is propellant free. An aerosol generating device generates an aerosol by passing liquid aerosol formulation through a flow passage heated to convert at least some of the liquid into a vapor, which is mixed with air to form an aerosol. In some embodiments, particles of the aerosol consist essentially of the cromolyn sodium or cromolyn sodium in combination with an additive such as a surfactant and/or low volatility liquid. The aerosol generator can be incorporated in a hand held inhaler and the aerosol can be delivered to a targeted portion of the lung using the inhaler for treatment of asthma.

This application claims priority under 35 U.S.C. §119 to U.S. Provisional Application No. 60/468,266 entitled AEROSOLIZATION OF AN ETHANOL-BASED SUSPENSION BY THE CAPILLARY AEROSOL GENERATOR filed on May 7, 2003, the entire content of which is hereby incorporated by reference.

### FIELD OF THE INVENTION

The invention relates generally to aerosol generation. More specifically, the invention relates to liquid aerosol formulations, aerosol generating devices and methods for generating aerosols.

### BACKGROUND OF THE INVENTION

Aerosols are gaseous suspensions of fine solid or liquid particles. Aerosols are useful in a wide variety of applications. For example, medicated liquids may be administered in aerosol form. Medicated aerosols include materials that are useful in the treatment of respiratory ailments. In such applications, the aerosols may be produced by an aerosol generator and inhaled into a patient's lungs. Aerosols are also used in non-medicinal applications including, for example, industrial purposes.

Aerosol generators are known that include a heated tube for vaporizing liquid. For example, commonly-assigned U.S. Pat. No. 5,743,251, which is incorporated herein by reference in its entirety, discloses an aerosol generator including a tube and a heater operable to heat the tube to a sufficient temperature to volatilize liquid in the tube. It is disclosed that the volatilized material expands out of an end of the tube and admixes with ambient air, thereby forming an aerosol.

As shown in FIG. 1, an aerosol generator 21 disclosed in U.S. Pat. No. 5,743,251 includes a tube 23 defining a capillary sized fluid passage and having an open end 25. A heater 27 is positioned adjacent to the tube 23. The heater 27 is connected to a power supply 29. The tube 23 also includes an inlet end 31 in fluid communication with a source 33 of liquid material. In operation, liquid is introduced into the tube 23. The heater 27 heats a portion of the tube 23 to a sufficiently high temperature to volatilize the liquid. The volatilized material expands out of the open end 25 of the tube and admixes with ambient air.

Other aerosol generators including a heated tube for vaporizing liquids to produce an aerosol are described in commonly-assigned U.S. Pat. Nos. 6,234,167; 6,568,390 and U.S. patent application Ser. Nos. 09/956,966 filed Sep. 21, 2001 and Ser. No. 10/003,437 filed Dec. 6, 2001, each incorporated herein by reference in its entirety.

### SUMMARY

Liquid aerosol formulations for producing cromolyn sodium aerosols having a desired particle size are provided. In addition, aerosol generating devices and methods for generating cromolyn sodium aerosols are provided.

An embodiment of a liquid aerosol formulation for producing an aerosol comprises a liquid carrier and cromolyn sodium. In preferred embodiments, the liquid carrier is a high volatility liquid such as ethanol, which can be heated to form a vapor, which does not form an appreciable condensation aerosol when the vapor is admixed with cooler air. That is, the vapor remains substantially in vapor form when admixed with the cooler air. However, the formulation can include other components such as surfactant and low volatility liquid additions. Particles of the cromolyn sodium form an aerosol when the vapor is admixed with air. Consequently, the resulting aerosol formed by vaporizing the liquid aerosol formulation and then admixing the vapor with air comprises aerosol particles that are substantially particles of only the cromolyn sodium or cromolyn sodium in combination with one or more additives such as a surfactant and/or low volatility liquid such as propylene glycol.

In a further preferred embodiment, the liquid aerosol formulation is propellant free. Further, the liquid aerosol formulation is preferably a suspension, emulsion or dispersion.

An embodiment of an aerosol generating device for generating an aerosol comprises a liquid source and a flow passage in fluid communication with the liquid source. The liquid source contains a liquid aerosol cromolyn sodium formulation including a carrier and a cromolyn sodium. In a preferred embodiment, the carrier includes at least one high volatility carrier. A heater is disposed to heat liquid in the flow passage to produce vapor. The vapor exits an outlet end of the flow passage and is admixed with air to produce an aerosol. In a preferred embodiment, the aerosol comprises aerosol particles that are substantially only the cromolyn sodium or cromolyn sodium in combination with at least one additive.

An exemplary embodiment of a method of generating an aerosol comprises supplying a liquid comprising a carrier and cromolyn sodium to a flow passage; and heating liquid in the flow passage to produce a vapor, which exits the flow passage. The vapor is admixed with air to produce an aerosol with a desired particle size. In a preferred embodiment, the aerosol particles are substantially only the cromolyn sodium and the carrier comprises a high volatility carrier. In another embodiment, the aerosol particles comprise cromolyn sodium and at least one additive, e.g., a surfactant and/or low volatility liquid such as propylene glycol.

An embodiment of a method of treating asthma in a subject in need thereof comprises administering a liquid aerosol cromolyn sodium formulation comprising cromolyn sodium to the subject. An embodiment of the method of treating asthma in a subject in need thereof, comprises administering an aerosol produced by vaporizing a liquid aerosol cromolyn sodium formulation generated by an aerosol generating device.

### BRIEF DESCRIPTION OF THE DRAWINGS

FIG. 1 illustrates an aerosol generator having a heated capillary passage according to the prior art.

FIG. 2 is a perspective view of an embodiment of an aerosol generating device with the cap removed.

FIG. 3 shows the aerosol generating device of FIG. 2 with the cap installed.

FIG. 4 illustrates an embodiment of an aerosol generating device.

FIG. 5 illustrates an embodiment of the fluid delivery assembly of the aerosol generating device.

FIG. 6 illustrates an embodiment of the capillary passage including two electrodes.

FIG. 7 is a graph illustrating the solubility of cromolyn sodium in various propylene glycol/ethanol (PG/EtOH) mixtures, with 0.25% Brij30 and 0.25% lecithin.

FIG. 8 is a graph illustrating the effect of storage time on the volume median diameter (VMD) of cromolyn sodium particles in the suspensions.

FIG. 9 is a graph illustrating the aerodynamic size distribution of an aerosol generated from a 1% w/v cromolyn sodium formulation (n=3).

FIG. 10 is a graph illustrating the metered and emitted dose fractions of a 1% w/v cromolyn sodium formulation (n=15).

FIG. 11 is a graph illustrating the effect of API concentration on the MMAD of cromolyn sodium aerosols (n=3).

FIG. 12 is a graph illustrating the respirable fraction (% of emitted dose, less than 5.6 μm) of cromolyn sodium aerosols as a function of API concentration (n=3).

FIG. 13 is a scanning electron microscope (SEM) image of cromolyn sodium particles in aerosol.

### DETAILED DESCRIPTION

Liquid aerosol cromolyn sodium formulations, aerosol generating devices and methods for generating aerosols from the liquid aerosol formulations are provided.

The liquid aerosol cromolyn sodium formulations can produce aerosols having selected compositions and controlled particle sizes. The liquid aerosol cromolyn sodium formulations are suitable for drug delivery applications via inhalation, wherein the liquid aerosol formulations can be used to produce aerosols having a desirable mass median aerodynamic diameter (MMAD) for targeted delivery. For pulmonary delivery, particles of smaller size are desired than for tracheobronchial delivery or delivery to the oropharynx or mouth. In preferred embodiments, the liquid aerosol formulations can be used to produce aerosols having a controlled particle size that is effective to achieve pulmonary delivery of drug formulations.

The liquid aerosol formulations include at least one high volatility carrier and a second component preferably comprising cromolyn sodium. In a preferred embodiment, the carrier is a liquid and the cromolyn sodium is suspended in the carrier. The cromolyn sodium formulation can include one or more additives including surfactants and low volatility liquids. For example, the cromolyn sodium formulation can include up to 10 weight % of a surfactant and/or up to 30 volume % of a low volatility liquid. In other embodiments, the liquid aerosol formulation can be a dispersion or an emulsion.

In a preferred embodiment, the liquid aerosol formulation is propellant free, and the liquid aerosol formulation is vaporized by heating and aerosolized by contacting the resulting vapor with air. In a preferred embodiment, the air is ambient air.

As used herein, the term “high volatility carrier” denotes a liquid that has a boiling point higher than 25° C. and remains substantially in the vapor state when it is vaporized by heating and the resulting vapor is admixed with ambient air. However, the second component of the liquid aerosol formulation forms an aerosol when the liquid aerosol formulation is vaporized and admixed with ambient air. By combining at least one high volatility carrier and cromolyn sodium, in a preferred embodiment, the liquid aerosol formulations can be used to produce aerosols containing solid aerosol particles that are substantially particles of only the cromolyn sodium, i.e., aerosol particles that are substantially free of the high volatility carrier.

The high volatility carriers have a low boiling point. In a preferred embodiment, the high volatility carriers have a boiling point of 100° C. or less, where 100° C. is the boiling point of water at atmospheric pressure. A preferred high volatility carrier is ethyl alcohol (ethanol), which has a boiling point of about 78° C. at a pressure of 1 atmosphere. Ethanol can be used in combination with other liquids, e.g., ethanol/water solutions containing 1-20 volume % water. In other preferred embodiments, the liquid aerosol formulation can contain as the carrier about 20-80 volume % water and 80 to 20 volume % ethanol, or about 80-100 volume % water and up to 20 volume % ethanol. Ethanol is a Federal Drug Administration (FDA) accepted excipient in drug products administered via inhalation.

Ethanol and other suitable high volatility carriers can be used as carriers for liquid aerosol cromolyn sodium formulations, which form an aerosol when heated into a vapor state and the vapor is admixed with air in which the carrier is present substantially only in the vapor state, i.e, substantially no aerosol of the carrier is formed. Accordingly, the aerosol particles in such aerosols are substantially only particles of the cromolyn sodium. Ethanol is converted from a liquid to a vapor by heating the liquid aerosol formulation to a sufficiently high temperature. In a preferred embodiment, the concentration of ethanol in the aerosol produced from the liquid aerosol formulation is below the saturation limit of ethanol in air with which the ethanol is admixed so that ethanol vapor substantially does not convert to an aerosol. Consequently, ethanol remains substantially in the vapor phase when used to form aerosols for delivery via inhalation.

As described above, liquids other than ethanol that have a high volatility can be used as a carrier in the liquid aerosol formulations. In a preferred embodiment, a liquid carrier that has a high volatility, but is not an FDA accepted excipient in drugs administered via inhalation, can be used in the liquid aerosol formulations for applications other than delivering drugs via such inhalation. Such other high volatility liquids can include, but are not limited to, water, other alcohols, such as isopropanol, butanol and the like. These liquids can be used as a carrier in the liquid aerosol formulation to produce aerosols that contain solid aerosol particles that are substantially particles of only the cromolyn sodium of the liquid aerosol formulation.

In a preferred embodiment, the cromolyn sodium in the liquid aerosol formulation is used for the treatment of asthma. Cromolyn sodium forms a suspension in ethanol to form an ethanol/cromolyn sodium suspension at ambient conditions. Ethanol/cromolyn sodium formulations can be provided in different compositions. For example, an ethanol/1% cromolyn sodium suspension can be used to produce aerosols for delivering a therapeutically effective dose of cromolyn sodium via inhalation. The concentration of cromolyn sodium can be varied to control the amount of cromolyn sodium in such aerosols.

As mentioned above, the at least one high volatility carrier and cromolyn sodium can be provided in a suspension comprising solid particles in a liquid, i.e., solid particles of the cromolyn sodium in the high volatility liquid carrier. Such suspensions can be heated to form an aerosol that contains liquid and/or solid aerosol particles that are substantially particles of only the cromolyn sodium.

In a preferred embodiment, the liquid aerosol cromolyn sodium formulation is flowed through a capillary sized flow passage in which the liquid is heated to a sufficiently high temperature to vaporize the high volatility carrier. The vapor exits the flow passage and admixes with gas, typically ambient air, to produce an aerosol that preferably is substantially aerosol particles of the cromolyn sodium, which is inhaled by a user. The size of the aerosol particles thus produced can be controlled for delivery to the lung.

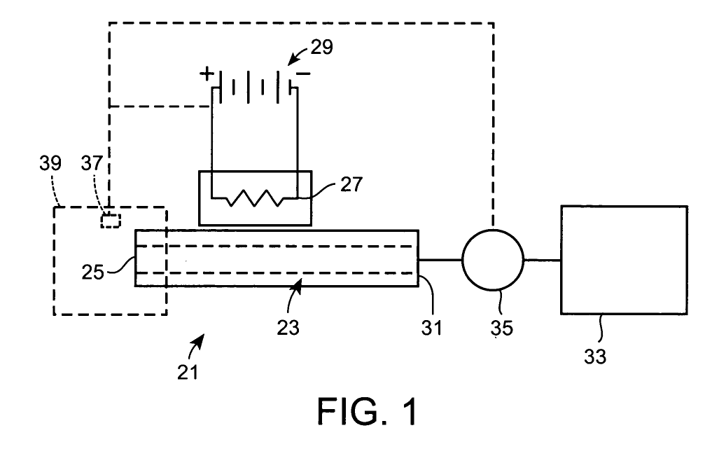

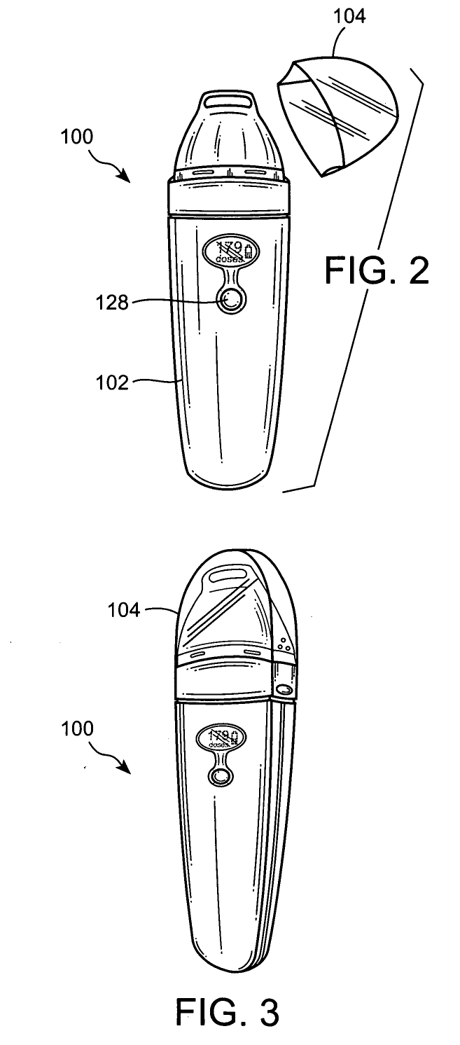

The high volatility liquid aerosol formulation can be aerosolized using the aerosol generator shown in FIG. 1. While any suitable aerosol generator can be used, FIGS. 2-4 illustrate an exemplary embodiment of a hand-held aerosol generating device 100 that can be used to produce aerosols of the liquid aerosol formulation for delivery via inhalation. The aerosol generating device 100 includes a housing 102; a removable protective cap 104, which activates a master on/off switch, (not shown); a fluid delivery assembly 110 including a liquid source 106 and a heater unit 130; a display 114; a battery unit 116; a charging jack 118; control electronics 120; a pressure sensor 122; an air inlet 124; a release 126 for detaching the fluid delivery assembly 110 from the aerosol generating device 100; a manually actuated master activation switch 128; an air passage 132 and a removable mouthpiece 134. FIG. 2 shows the cap 104 removed from the aerosol generating device 100, while FIG. 3 shows the cap installed.

In a preferred embodiment, the fluid delivery assembly 110 is removably attachable to a portion of the aerosol generating device 100 by any suitable attachment construction. For example, conductive contacts (not shown) can be provided in the aerosol generating device to make electrical contact with the heater unit 130, when the fluid delivery assembly 110 is attached to the aerosol generating device. In such embodiments, the fluid delivery assembly 110, which includes the wetted components of the aerosol generating device, can be replaced in the vapor generating device as a complete unit. As described below, the fluid delivery assembly 110 can provide aerosols having a controlled particle size. Different fluid delivery assemblies 110 that can provide aerosols having different compositions and/or particle sizes can be interchanged in the aerosol generating device.

The fluid delivery assembly 110 can be replaced after liquid contained in the liquid source 106 has been consumed. A fluid delivery assembly 110 including a liquid source containing the same or a different medicament, and that produces the same or a different aerosol particle size, can then be installed in the aerosol generating device.

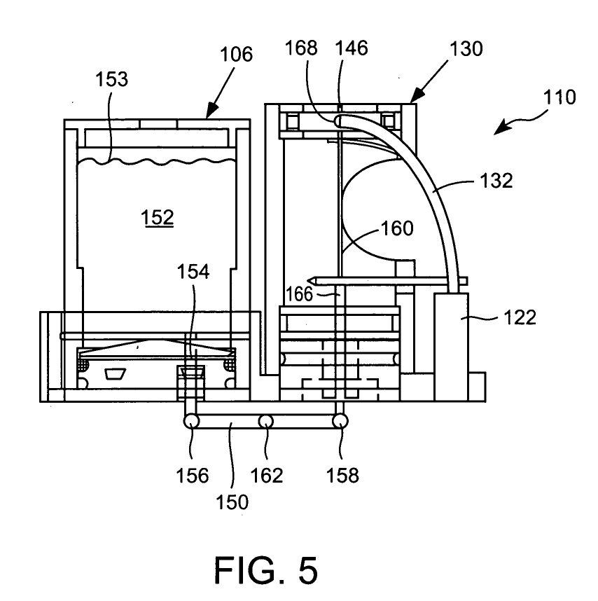

FIG. 5 illustrates a portion of the fluid delivery assembly 110, including a liquid source 106 and heater unit 130. Liquid is supplied from the liquid source 106 to the heater unit 130 through a flow passage 150.

The liquid source 106 comprises a reservoir 152 for containing a volume of liquid 153. In an embodiment, the liquid source 106 has a liquid capacity for delivering a selected number of doses of a selected volume. For example, the doses can be 5 μl doses and the reservoir 152 can be sized to contain multiple doses. Preferably, the liquid source can contain from about 10 doses to about 500 doses, e.g., 50 to 250 doses. However, the dose capacity of the liquid source can be determined by the desired application of the aerosol generating device. The liquid contained in the liquid source can be any liquid aerosol formulation that can be vaporized and aerosolized in the aerosol generating device to produce a desired aerosol as described above. In a preferred embodiment, the liquid contains cromolyn sodium formulated to be inhaled into the user's lungs in aerosol form.

The liquid source 106 includes a flow passage 154, which provides fluid communication from the reservoir 152 to the flow passage 150. The aerosol generating device 100 includes at least one valve disposed to control flow of the liquid from the liquid source 106 into the heater unit 130. For instance, the aerosol generating device may include a single valve (not shown) to control flow of the liquid in the flow passage, or a plurality of valves. In a preferred embodiment, the aerosol generating device includes an inlet valve 156 and an outlet valve 158. The inlet valve 156 is operable to open and close an inlet of the flow passage 150, which controls the supply of liquid from the liquid source 106 into the flow passage 150. The outlet valve 158 is operable to open and close an outlet end of the flow passage 150, which controls the supply of liquid from the flow passage 150 into a heated flow passage.

The aerosol generating device 100 preferably includes a metering chamber 162 located in the flow passage 150 between the inlet valve 156 and the outlet valve 158. The metering chamber 162 is preferably sized to contain a predetermined volume of the liquid, such as a volume of the liquid that corresponds to one dose of the aerosolized cromolyn sodium. A discharge member 164 can be used to open the metering chamber 162 during a liquid filling cycle, and to empty the metering chamber during a liquid delivery cycle, as described in greater detail below.

The heater unit 130 of the fluid delivery assembly 110 comprises a heated flow passage 160. The flow passage 160 is preferably a capillary sized flow passage, referred to hereinafter as a “capillary passage.” The capillary passage 160 forms a portion of the entire flow passage in the aerosol generating device 100. The capillary passage 160 includes an open inlet end 166, and an opposite open outlet end 168. During operation of the aerosol generating device 100, liquid is supplied into the capillary passage 160 at the inlet end 166 from the flow passage 150.

The capillary passage 160 can have different transverse cross-sectional shapes. Different portions of the capillary passage can have different cross-sectional shapes. As described below, the size of the capillary passage 160 can be defined by its transverse cross-sectional area. For example, the capillary passage can have a maximum transverse dimension of 0.01 to 10 mm, preferably 0.05 to 1 mm, and more preferably 0.1 to 0.5 mm. Alternatively, the capillary passage can be defined by its transverse cross sectional area, which can be 8×10⁻⁵ to 80 mm², preferably 2×10⁻³ to 8×10⁻¹ mm², and more preferably 8×10⁻³ to 2×10⁻¹ mm².

As described in commonly-assigned U.S. patent application Ser. No. 10/655,017, filed Sep. 5, 2003, which is incorporated herein by reference in its entirety, embodiments of the capillary passage 160 can comprise an outlet section, which controls the velocity of vapor exiting the outlet end 168 of the capillary passage, i.e, the exit velocity of the vapor, so as to control the particle size of aerosol generated by the aerosol generating device 100.

The material forming the capillary passage can be any suitable material, including metals, plastics, polymers, ceramics, glasses, or combinations of these materials. Preferably, the material is a heat-resistant material capable of withstanding the temperatures and pressures generated in the capillary passage, and also resisting the repeated heating cycles utilized to generate multiple doses of aerosols. In addition, the material forming the capillary passage preferably is non-reactive with the liquid that is aerosolized.

In another alternative embodiment, the capillary passage can be formed in a polymer, glass, metal and/or ceramic monolithic or multilayer (laminated) structure (not shown). Suitable ceramic materials for forming the capillary passage include, but are not limited to, alumina, zirconia, silica, aluminum silicate, titania, yttria-stabilized zirconia, or mixtures thereof. A capillary passage can be formed in the monolithic or multilayer body by any suitable technique, including, for example, machining, molding, extrusion, or the like.

In embodiments, the capillary passage can have a length from 0.5 to 10 cm, and preferably from 1 to 4 cm.

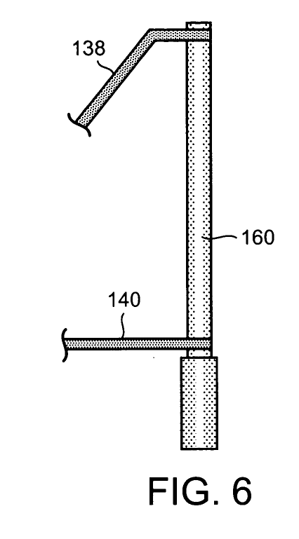

The fluid supplied from the liquid source 106 is heated in the capillary passage to form a vapor during operation of the aerosol generating device 100. However, the liquid formulation need not be entirely vaporized. In a preferred embodiment shown in FIG. 6, the capillary 160 comprises metal tubing heated by passing an electrical current along a length of the capillary via a first electrode 138 and a second electrode 140. However, as described above, the capillary passage can have other alternative constructions, such as a monolithic or multi-layer construction, which include a heater such as a resistance heating material positioned to heat the fluid in the capillary passage. For example, the resistance heating material can be disposed inside of, or exterior to, the capillary passage.

The capillary passage 160 may comprise an electrically conductive tube provided with the electrode 138, which is the downstream electrode, and the electrode 140, which is the upstream electrode. Electrode 140 is preferably made of copper or a copper-based material, while electrode 138 preferably is made of a higher resistance material, such as stainless steel. In this embodiment, the capillary 160 is a controlled temperature profile construction, such as disclosed in copending and commonly assigned U.S. Pat. No. 6,640,050, issued Oct. 28, 2003, which is incorporated herein by reference in its entirety. In the controlled temperature profile capillary, the electrode 138 has an electrical resistance sufficient to cause it to be heated during operation of the aerosol generating device, thereby minimizing heat loss at the outlet end of the capillary tube.

The tube forming the capillary passage can be made entirely of stainless steel or any other suitable electrically conductive materials. Alternatively, the tube can be made of a non-conductive or semi-conductive material incorporating a heater made from an electrically conductive material, such as platinum. Electrodes connected at spaced positions along the length of the tube or heater define a heated region between the electrodes. A voltage applied between the two electrodes generates heat in the heated region of the capillary passage based on the resistivity of the material(s) making up the tube or heater, and other parameters such as the cross-sectional area and length of the heated region section. As the fluid flows through the capillary passage into the heated region between the first and second electrodes, the fluid is heated and at least some of the fluid is converted to a vapor. The vapor passes from the heated region of the capillary passage and exits from the outlet end. In some preferred embodiments, the volatilized fluid is entrained in ambient air as the volatilized fluid exits from the outlet, causing the volatilized fluid to form an aerosol. In a preferred embodiment, the MMAD of the aerosol particles is 0.5 to 5 μm.

The temperature of the liquid in the capillary passage can be calculated based on the measured or calculated resistance of the heating element. For example, the heating element can be a portion of a metal tube, or alternatively a strip or coil of resistance heating material. Control electronics can be used to regulate the temperature of the capillary passage by monitoring the resistance of the heater.

Resistance control can be based on the simple principle that the resistance of the heater increases as its temperature increases. As power is applied to the heating element, its temperature increases because of resistive heating and the actual resistance of the heater also increases. When the power is turned off, the temperature of the heater decreases and correspondingly its resistance decreases. Thus, by monitoring a parameter of the heater (e.g., voltage across the heater using known current to calculate resistance) and controlling application of power, the control electronics can maintain the heater at a temperature that corresponds to a specified resistance target. The use of one or more resistive elements could also be used to monitor temperature of the heated liquid in cases where a resistance heater is not used to heat the liquid in the capillary passage.

The resistance target is selected to correspond to a temperature that is sufficient to cause heat transfer to the liquid such that at least some of the liquid is volatilized and expands out the open end of the capillary passage. The control electronics activates the heating, such as by applying for a duration of time, pulsed energy to the heater and after and/or during such duration, determines the real time resistance of the heater, using input from the measuring device. The temperature of the heater can be calculated using a software program designed to correlate measured resistance of the heater. In this embodiment, the resistance of the heater is calculated by measuring the voltage across a shunt resistor (not shown) in series with the heater (to thereby determine current flowing to the heater) and measuring the voltage drop across the heater (to thereby determine resistance based on the measured voltage and current flowing through the shunt resistor). To obtain continuous measurement, a small amount of current can be continually passed through the shunt resistor and heater for purposes of making the resistance calculation and pulses of higher current can be used to effect heating of the heater to the desired temperature.

If desired, the heater resistance can be derived from a measurement of current passing through the heater, or by other techniques used to obtain the same information. The control electronics determines whether or not to send an additional duration of energy based on the difference between desired resistance target for the heater and the actual resistance as determined by control electronics.

In a developmental model, the duration of power supplied to the heater was set at 1 millisecond. If the monitored resistance of the heater minus an adjustment value is less than the resistance target, another duration of energy is supplied to the heater. The adjustment value takes into account factors, such as, for example, heat loss of the heater when not activated, the error of the measuring device and the cyclic period of the controller and switching device. In effect, because the resistance of the heater varies as a function of its temperature, resistance control can be used to achieve temperature control.

In embodiments, the capillary passage 160 can be constructed of two or more pieces of 32 gauge, 304 stainless steel tubing. In this embodiment, the downstream electrode can be a 3.5 mm length of 29 gauge tubing, while the upstream electrode may have any geometry that minimizes the resistance of the electrode, such as gold (Au) plated copper (Cu) pins.

The control electronics 120 can control the temperature of the capillary passage 160 by monitoring the resistance of the heater used to heat the capillary passage 160. In an embodiment, the control electronics 120 measures voltage and current in order to calculate the resistance across a length of the capillary passage 160. If the control electronics determines that the resultant resistance is below the target value, the control electronics turns power on for a selected period of time. The control electronics continues to repeat this process until the target resistance for the capillary passage 160 is reached. Likewise, if the control electronics determines that the resistance is higher than required for the temperature of the capillary passage 160, the control electronics turns off power for a selected period of time.

In this embodiment, the control electronics 120 may include any processor capable of controlling the resistance of the capillary passage 160 via the electrodes 138 and 140, such as a microchip PIC16F877, available from Microchip Technology Inc., located in Chandler, Ariz., which is programmed in assembly language.

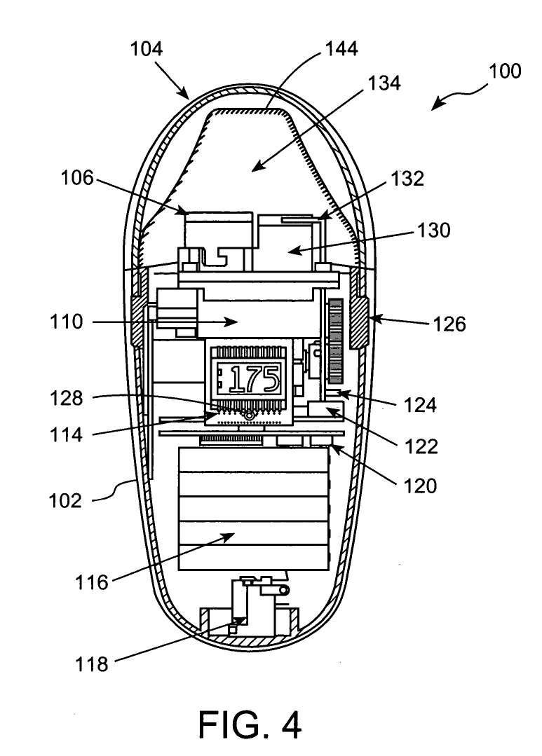

As shown in FIGS. 4 and 5, the pressure sensor 122 is in fluid communication with the mouthpiece 134 via the air passage 132. The air passage 132 includes the air inlet 124 through which ambient air within the housing is drawn into the air passage 132 by a user inhaling on the mouthpiece 134. In a preferred embodiment, the aerosol generating device 100 is activated by a user inhaling on an outlet 144 of the mouthpiece 134. This inhalation causes a differential pressure in the air passage 132, which is sensed by the pressure sensor 122. The pressure sensor 122 can be extremely sensitive. For example, the pressure sensor can be triggered at a selected threshold value of air flow through the air passage 132, for example, as low as about 3 liters/min. This value equals less than about 1/10 of the typical human inhalation flow rate. Accordingly, the user can trigger the pressure sensor without wasting appreciable lung volume.

Alternatively, the fluid delivery assembly 110 can be activated by a user manually depressing the switch 128.

The pressure sensor 122 or switch 128 activates the fluid delivery assembly 110 to cause liquid 153 (e.g., a liquid aerosol formulation including a high volatility carrier and a drug) to flow from the liquid source 106 to the capillary passage 160 of the heater unit 130. The fluid is heated in the capillary passage 160 by the heater to a sufficiently high temperature to vaporize at least some or substantially all of the liquid. Ambient air is delivered through the air passage 132 to an entrainment region 146 proximate to the outlet end of the capillary passage, at which the vapor is admixed with the ambient air to produce an aerosol.

In alternative embodiments, a pressurized air source can be used with the aerosol generating device to provide dilution air to mix with the aerosol. For example, the pressurized air source can be a compressed air source located within the aerosol generating device (not shown), a fan/blower to flow air into the mouthpiece, or any other suitable device.

The control electronics 120 can perform various selected functions in the aerosol generating device 100. For example, the control electronics 120 can control the temperature profile of the capillary passage 160 during operation of the aerosol generating device 100. The control electronics 120 can also control the output of the display 114. The display is preferably a liquid crystal display (LCD). The display can depict selected information pertaining to the condition or operation of the aerosol generating device 100. The control electronics can also control the operation of the inlet valve 156, discharge member 164 and outlet valve 158 during operation of the aerosol generating device 100; monitor the initial pressure drop caused by inhalation and sensed by the pressure sensor 122; and monitor the condition of the battery unit 116 that provides electrical power to components of the aerosol generating device.

In the embodiment shown in FIG. 4, the battery unit 116 can be, for example, a rechargeable battery. The battery unit is preferably rechargeable via the charging jack 118. The battery unit provides power to components of the aerosol generating device (e.g., the control electronics 120, pressure sensor 122, etc.) and the master on/off switch.

The master on/off switch controls powering up and powering down of the aerosol generating device 100 during operation. The master on/off switch also activates the display 114. In an embodiment, the display provides information including, for example, the number of doses remaining within the liquid source 106, a failure of the heater unit 130, and a detected low voltage condition of the battery unit 116. The control electronics 120 can also include functionality via the processor for displaying the number of remaining doses, information on patient compliance, lockout times and/or child safety locks.

During operation of the aerosol generating device 100, a user removes the cap 104 to activate components of the aerosol generating device and expose the mouthpiece 134. The user activates switch 128, or inhales on the mouthpiece, which creates a pressure drop in the interior of the mouthpiece. This pressure drop is detected by the pressure sensor 122, which then sends a signal to a controller included in the control electronics 120, which operates the fluid delivery assembly 110.

The metering chamber 162 is filled and emptied by actuation of the discharge member 164. Closing of the discharge member 164 with the inlet valve 156 closed and the outlet valve 158 opened empties liquid in the metering chamber 162, which forces liquid present in the flow passage 150 downstream of the metering chamber into the capillary passage 160. The metering chamber 162 ensures that a desired volume of liquid in aerosol form is delivered by the aerosol generating device 100 to the user. The metering chamber can have a selected dose volume of, e.g., 5 μl. However, the metering chamber can have any desired volume depending upon the application of the aerosol generating device 100. After delivery of the desired volume of the medicament to the capillary passage 160, the outlet valve 158 is closed, and the flow passage 150 is refilled with liquid from the liquid source 106.

During a fill cycle of the aerosol generating device 100, the metering chamber 162 is filled with liquid from the liquid source 106. The inlet valve 156 is opened and the outlet valve 158 is closed, while the discharge member 164 is opened to allow the liquid to fill the metering chamber 162.

During delivery of the liquid to the capillary passage 160, the inlet valve 156 is closed. As the inlet valve 156 closes, the outlet valve 158 is opened, while the discharge member 164 is closed to empty the metering chamber 162 and force liquid from the flow passage 150 into the heated capillary passage 160.

Liquid flows through the heated capillary passage 160 and at least some or substantially all of the liquid exits as a vapor. At the exit of the capillary passage 160, ambient air provided via the air passage 132 admixes with vapor in the entrainment region 146 to form the aerosol. For example, the liquid aerosol formulation may include low volatility liquid such as PG which can be jetted from the flow passage as an aerosol of liquid particles of PG containing a medicament such as cromolyn sodium.

Preferably, the aerosol particles have a MMAD between about 0.5 μm and about 5 μm. However, if desired, the aerosol particles can have a smaller particle size, such as an MMAD of less than 0.5 μm, for example, less than 0.1 μm. As described above, the aerosol generating device can provide aerosols having a controlled particle size, including aerosols sized for the targeted delivery of drugs to the lung. These aerosols offer a number of advantages for delivering drugs to the deep lung. For example, mouth and throat deposition are minimized, while deposition in the deep lung is maximized, especially when combined with a breath hold.

The aerosol generating device preferably generates cromolyn sodium aerosols in which 95% of the aerosol particles have a size in the range between about 0.5 μm to about 5 μm. If desired, the aerosol formulation may include a low volatility liquid in an amount effective to achieve a desired MMAD. For example, up to 30 volume % PG can be added to increase the MMAD of the aerosol. The aerosol generating device preferably incorporates a processor chip for controlling the generation process. The processor, with suitable sensors, also triggers the aerosol generation at any desired time during an inhalation.

Operation of the preferred aerosol generating device for delivering aerosolized medicaments is as follows. First, the liquid aerosol formulation containing at least one high volatility liquid carrier and medicament is delivered to the heated capillary passage. The liquid at least partially vaporizes in the capillary passage and exits as a vapor jet from the open end of the capillary passage. The vapor jet entrains and mixes with ambient air and forms a highly concentrated, fine aerosol. As described above, application of heat to vaporize the liquid is preferably achieved by resistive heating from passing an electric current through the heater. The applied power is adjusted to achieve desired degree of conversion of the fluid into a vapor.

The aerosol generating device can form aerosols over a range of fluid flow rates dependent on the size of the capillary passage and the power available to vaporize the liquid.

As will be appreciated, the aerosol generating device is capable of controlled vaporization and aerosol formation of drug formulations. The aerosol generating device can provide immediate delivery of aerosol to a patient, thereby not wasting lung capacity, which may be limited due to the health of the patient. Also, the aerosol generating device can provide consistent delivery of controlled amounts of drug formulation to a patient. In addition, in preferred embodiments, the aerosol generated by the aerosol generating device including a capillary passage is only slightly affected by relative humidity and temperature.

In a preferred embodiment, the emitted dose (i.e., the aerosolized dose) can be at least about 85%, preferably about at least 85%-95%, of the metered dose of the liquid used to produce the aerosol; the respirable fraction of the emitted dose can be at least 30%, preferably at least 50%, more preferably at least 80%, e.g., about 85%-95%, of the emitted dose; and the variation in repeated delivery of the emitted dose can be less than about 10%, preferably less than 5%.

### EXAMPLES

### Example 1

The physical stability of cromolyn sodium (an antihistamine) suspended in ethanol was evaluated. Cromolyn sodium was selected as a model compound for suspension testing because it is virtually insoluble in ethanol, and the required dosage strength is relatively high. It is currently used for prophylactic treatment of asthma.

Commercially available micronized cromolyn sodium was suspended in absolute ethanol at 5% w/v. Surfactants with a wide range of hydrophilic-lipophilic balance (HLB) (Table 1) were separately added to aliquots of the suspension at 1% w/v, and sonnicated for 2 minutes. The suspensions were allowed to stand in separate vials for 24 hours. Photographs were taken of the vials after mixing, after settling for 5 minutes, after settling for 15 minutes, after settling for 2 hours, after settling for 18 hours and after settling for 24 hours. Based on the results of these measurements, Table 2 sets forth the rank order of particle settling for the five suspensions.

TABLE 1
Surfactants Used in Cromolyn Sodium Microsuspensions

| Suspension | | | |
|---|---|---|---|
| No. | Surfactant (Trade Name) | HLB | Type |
| 1 | Sorbitan Trioleate (Span 85) | 1.8 | Non-ionic |
| 2 | Polyoxyethylene Laurel Ether (Brij 30) | 9.5 | Non-ionic |
| 3 | Polyoxyethylene Sorbitan Monooleate | 15.0 | Non-ionic |
| | (Tween 80) | | |
| 4 | Oleic Acid | 1.0 | Anionic |
| 5 | None | N/A | N/A |

TABLE 2
Order of Particle Settling of Cromolyn Sodium
(Suspended in Ethanol) as a Function of Surfactant

| Suspension No. | Surfactant | Rank Order of Particle Settling* |
|---|---|---|
| 1 | Span 85 | 5 |
| 2 | Brij 30 | 4 |
| 3 | Tween 80 | 1 |
| 4 | Oleic Acid | 3 |
| 5 | None | 2 |

*1-fastest, 5-slowest

Suspensions 3 and 5 began to settle after five minutes. Suspensions 1, 2 and 4 began settling after two hours of standing. Complete settling was achieved after 18 hours of standing. The stability of the suspensions appeared to relate to the HLB of the surfactant. Sorbitan trioleate and oleic acid, both of which have low HLB values, were most effective in stabilizing cromolyn sodium particles in ethanol. This is most likely related to the high surface activities of Span 85 and oleic acid. Because of the non-polar nature of Span 85 and oleic acid (as indicated by low HLB values), both surfactants prefer to concentrate at the particle-ethanol interface rather than interact with the more polar ethanol in the liquid phase. Thus, Span 85, Brij 30 and oleic acid all acted as stabilizers for cromolyn sodium-ethanol suspensions. In contrast, the addition of Tween 80 to the cromolyn sodium-ethanol suspension not only did not impart additional stability of the suspension, but also further accelerated the settling of cromolyn sodium particles. The instability of the suspension in the presence of Tween 80 stemmed from a rapid flocculation of cromolyn sodium particles after sonication and resulted in a high sedimentation volume. Hence, Tween 80 behaved as a flocculating agent in the cromolyn sodium-ethanol suspension.

After 24 hours of standing at ambient laboratory conditions, all but suspension 3 (Tween 80) formed hard cakes. While suspension 3 required only inversions for a complete resuspension, all others required more than 10 inversions. The more rapid resuspension of suspension 3 is most likely due to the high degree of flocculation of drug particles induced by Tween 80 in suspension 3.

The stability of cromolyn sodium-ethanol suspension formulations was further evaluated using Lecithin and Brij 30 in the suspensions to prevent caking and to maintain the formulations well suspended. Zeta potential measurements were taken and formulation re-suspendability (number of inversions required for remixing) was assessed. The results are summarized in Table 3.

All formulations were well suspended for at least two hours after mixing. After 24 hours, all formulations were completely sedimented. While formulations 5 and 6 settled to form loose agglomerates and required only 4 to 5 gentle inversions to resuspend, most others formed hard cakes and were extremely difficult to resuspend. Only formulations 5 and 8 had zeta potentials that are in the range that is generally considered as an optimal range (−50 to −20 mV).

TABLE 3
Zeta Potentials and the Resuspendability of
Cromolyn Sodium Suspensions

| | | | | |
|---|---|---|---|---|
| | | % | Zeta Potential | # Inversions |
| Formulation No. | % Brij 30 | Lecithin | (mV) | to resuspend |

| | | | | |
|---|---|---|---|---|
| 1 | 0 | 0 | −51 | 0 |
| 2 | 0 | 0.5 | −9 | >50 |
| 3 | 0 | 1.0 | −5 | >50 |
| 4 | 0.5 | 0 | −53 | 0 |
| 5 | 0.5 | 0.5 | −31 | 5 |
| 6 | 0.5 | 1.0 | −4 | 4 |
| 7 | 1.0 | 0.5 | −3 | 34 |
| 8 | 1.0 | 0 | −40 | 0 |

* Required vigorous shaking to resuspend.

The foregoing data demonstrates that micronized cromolyn sodium can be dispersed in absolute ethanol at 1% w/v with the aid of lecithin (a zwitterionic surfactant) and non-ionic surfactants. The physical stability of the formulations was sufficient for forming aerosols through vaporization of the liquid formulations. For example, a suspension formulation of cromolyn sodium in ethonol containing lecithin and Brij 30 exhibited good short-term stability. There was no visible settling of drug particles in the formulation in the first hour after mixing. The aerosols of this formulation generated by a capillary aerosol generator had aerosol MMAD particle sizes in the range of 1-5 microns. The particle size appeared to be dependent on the size of the micronized cromolyn sodium.

### Example 2

The feasibility of generating inhalable aerosols from ethanol-based suspension formulations by the CAG was evaluated.

Cromolyn sodium (CrNa) formulations were prepared by milling the active pharmaceutical ingredient (API) to 0.5 μm in propylene glycol (PG)/ethanol mixtures using a centrifugal ball mill (Retsch, Germany). The nominal concentration of cromolyn sodium in the formulation ranged from 0.75 to 3% (w/v). The vehicles were prepared from absolute ethanol and propylene glycol in PG/ethanol ratios ranging from 15/85 to 30/70 v/v. PG was added to each formulation to prevent capillary clogging. The required concentration was dependent on drug concentration and is shown in Table 4. In addition, Brij 30 and lecithin were added to the formulations at 0.25% w/v as stabilizers.

Aerosols were generated using a 26 gauge, 25 mm stainless steel capillary tube by heating the capillary to a tip temperature of approximately 100° C. The formulation was pumped through the capillary at a volumetric flow rate of 5 μl/second for a duration of 10 seconds. The size distribution of the aerosols were analyzed by sampling into a MOUDI Cascade Impactor (MSP Corp., Minnesota) operating at 30 L/minute. The emitted dose was determined by sampling the delivered dose into a USP emitted dose apparatus. The particle shape and morphology of the CrNa aerosol were evaluated by scanning electron microscopy.

TABLE 4
Formulation composition of cromolyn sodium suspensions

| Nominal | | | | |
|---|---|---|---|---|
| Concentration | | | | |
| of Cromolyn | PG | EtOH | Brij 30 | Lecithin |
| Sodium | (% v/v) | (% v/v) | (% w/v) | (% w/v) |
| 0.75 | 10 | 90 | 0.25 | 0.25 |
| 1.0 | 15 | 85 | 0.25 | 0.25 |
| 1.5 | 20 | 80 | 0.25 | 0.25 |
| 2.0 | 25 | 75 | 0.25 | 0.25 |
| 3.0 | 30 | 70 | 0.25 | 0.25 |

The solubility of cromolyn sodium in the Table 4 PG/EtOH mixtures is sufficiently low that it can be considered almost insoluble. When formulated with 0.25% w/v Brij 30 and 0.25% lecithin, cromolyn sodium suspension formulations remained visually stable over a period of one to two weeks. There was no significant change in the particle size distribution of the suspended particles up to three days of standing at ambient condition. The addition of PG was primarily to prevent capillary clogging. The amount of PG required in the formulation was dependent on the API concentration. This appears to suggest that PG acts as a lubricant in preventing the cromolyn sodium particles from clogging during their transit through the capillary.

The CAG was able to aerosolize suspension formulations with drug loadings ranging from 0.75 to 3% w/v. On average, 97% of the nominal dose was metered into the capillary, and roughly 93% of the metered dose was emitted from the CAG. The average MMAD of the aerosols emitted was in the range of 3 to 5 μm. However, the majority of the emitted dose was lost in the USP port elbow (which mimics the throat of a subject inhaling the volatilized aerosol formulation) and only 30-40% of the emitted dose by mass was found below 5.6 μm, a size range generally considered suitable for lung deposition. The low respirable fraction is most likely due to the presence of PG in the formulation. It is possible that during its transit through the capillary that is being heated to 100° C., ethanol quickly evaporates from the formulation. Because of its high volatility, it is believed that PG does not significantly evaporate, but instead converts into fine mist and subsequently exits the capillary as an aerosol. The cromolyn sodium particles, of at least an order of magnitude lower in concentration and size, most likely remained suspended in the PG droplets. This results in the generation of cromolyn sodium containing aerosols of a much larger aerodynamic size. Thus, PG can be used to increase the MMAD of the aerosolized cromolyn sodium.

The capillary aerosol generator is capable of producing soft-mist aerosols with the characteristics desired for respiratory drug delivery from suspension formulations. Formulations with drug loading of up to 3% w/v can be consistently delivered by the CAG without capillary clogging or significant drug loss. The aerosol characteristics are relatively insensitive to the drug loading in the range of 1 to 3% w/v.

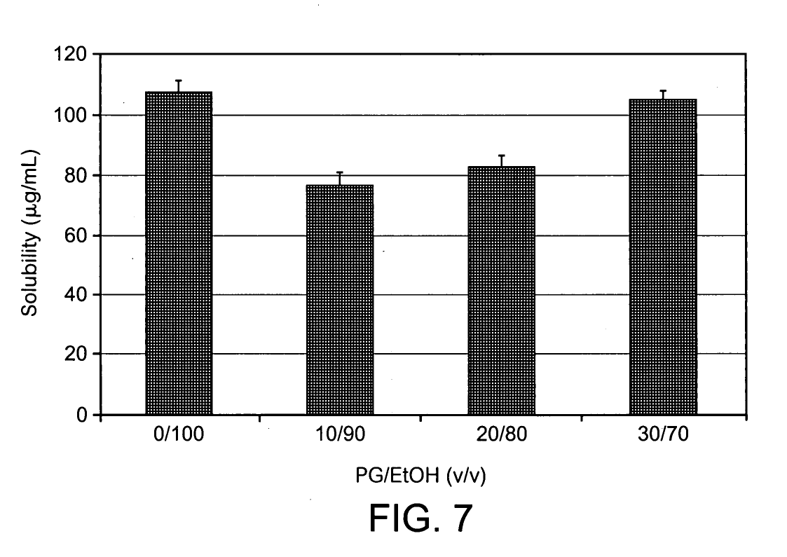

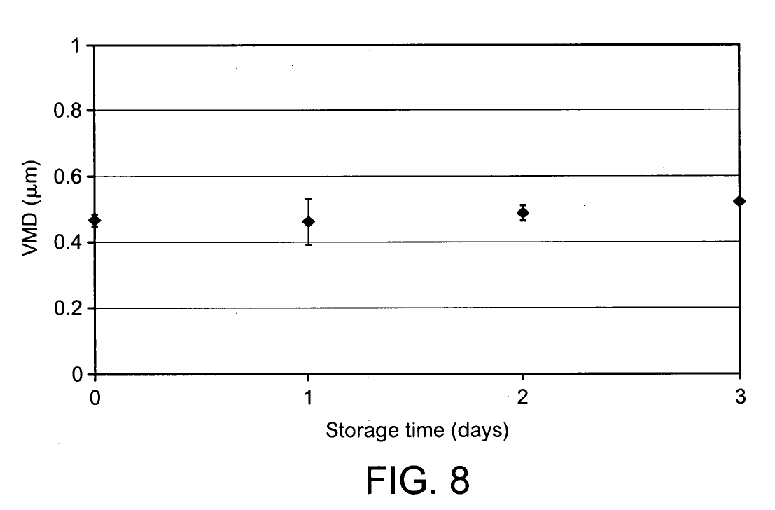

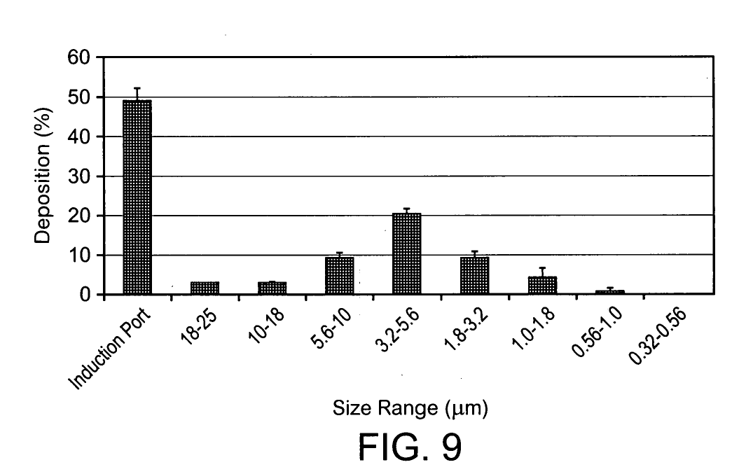

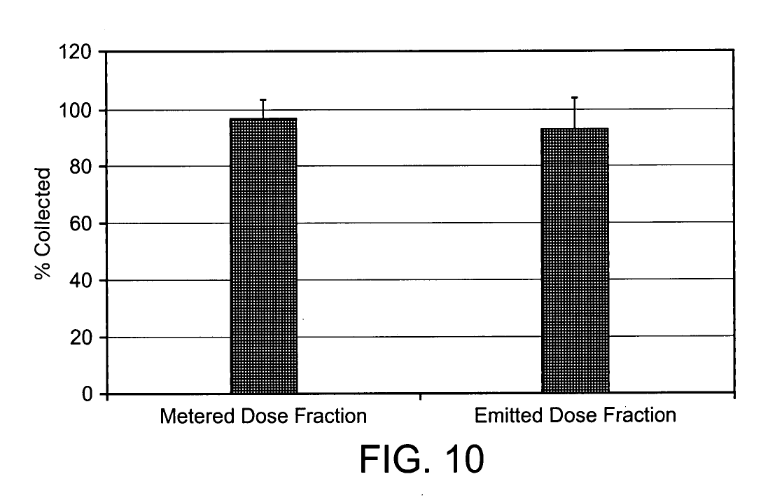

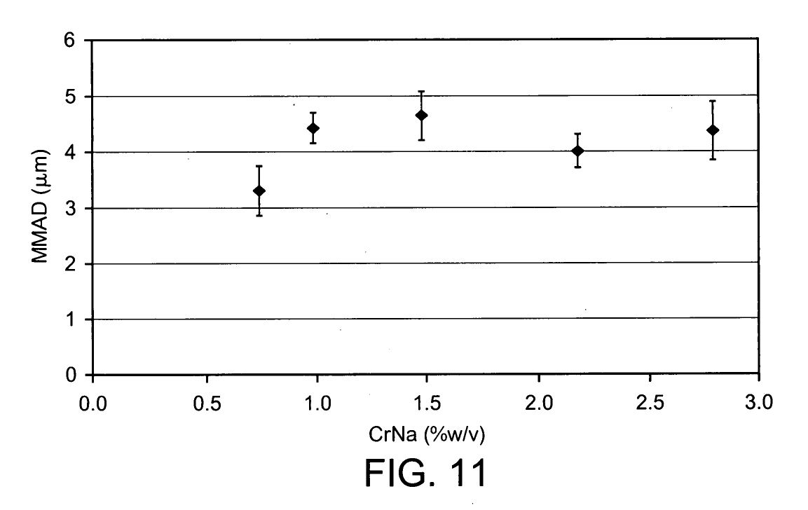

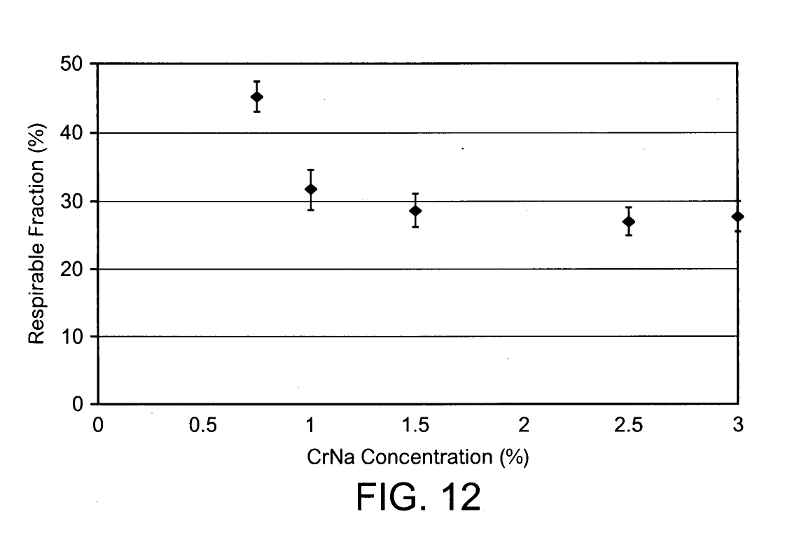

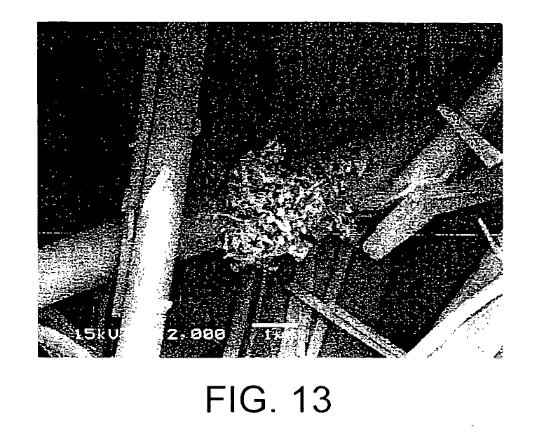

FIGS. 7-13 show results of measurements based on the forgoing examples. FIG. 7 is a graph illustrating the solubility of cromolyn sodium in various PG/EtOH mixtures, with 0.25% Brij30 and 0.25% lecithin. FIG. 8 is a graph illustrating the effect of storage time on the volume median diameter (VDM) of cromolyn sodium particles in suspensions. No significant change was observed in the size of the cromolyn sodium particles during three days of storage at ambient conditions. FIG. 9 is a graph illustrating aerodynamic size distribution of an aerosol generated with 1% w/v cromolyn sodium formulation (n=3). About 50% of the aerosol was deposited in the USP induction port and the majority of the cromolyn sodium aerosol collected on the impactor was in the respirable range (less than 5.6 microns). FIG. 10 is a graph illustrating the metered and emitted dose fractions of a 1% w/v cromolyn sodium formulation (n=15). On average, 97% of the nominal dose was metered into the capillary heater. About 93% of the metered dose was emitted from the aerosol generator. FIG. 11 is a graph illustrating the effect of API concentration on the MMAD of cromolyn sodium aerosols (n=3). At 1% cromolyn sodium concentration, the higher MMAD of the aerosols was generally independent of API concentrations. Aerosol particles generated were much larger than the suspended cromolyn sodium particles. FIG. 12 is a graph illustrating the respirable fraction (% of emitted dose less than 5.6 microns) of cromolyn sodium aerosols as a function of API concentration (n=3). Except at low API concentration, the respirable fraction was generally dependent on the concentration of cromolyn sodium. The CAG was capable of aerosolizing suspension formulations with cromolyn sodium loading up to 3% w/v. FIG. 13 is a scanning electron microscope (SEM) image of aerosolized cromolyn sodium particles.

The above-described exemplary modes of carrying out the invention are not intended to be limiting. It will be apparent to those of ordinary skill in the art that modifications thereto can be made without departure from the spirit and scope of the invention as set forth in the accompanying claims.

What is claimed is:

1. A liquid aerosol formulation adapted to form a vaporization aerosol, comprising ethanol and cromolyn sodium, the liquid aerosol formulation being propellant free.

2. The liquid aerosol formulation of claim 1, further comprising a surfactant having a hydrophilic-lipophilic balance of below 15 or below 10.

3. The liquid aerosol formulation of claim 1, wherein the surfactant is lecithin, sorbitan trioleate, oleic acid, polyoxyethylene laurel ether or mixtures thereof.

4. The liquid aerosol formulation of claim 1, further comprising up to 30 volume % of a low volatility liquid such as propylene glycol.

5. A propellant-free aerosol containing aerosol particles entrained in a vapor, wherein substantially all of the aerosol particles consist essentially of cromolyn sodium and surfactant and the vapor consists essentially of a carrier.

6. The aerosol of claim 5, wherein the surfactant is lecithin, sorbitan trioleate, oleic acid, polyoxyethylene laurel ether or mixtures thereof.

7. The aerosol of claim 5, wherein the aerosol particles further comprise a low volatility liquid such as propylene glycol.

8. An aerosol generating device, comprising:
a liquid source of a liquid aerosol formulation comprising ethanol and cromolyn sodium;
a flow passage in fluid communication with the liquid source; and
a heater disposed to heat liquid aerosol formulation in a heated portion of the flow passage to produce a vapor which admixes with air to produce an aerosol containing cromolyn sodium and optionally a surfactant and/or low volatility liquid such as propylene glycol.

9. A method of generating an aerosol, comprising:
(a) supplying a liquid aerosol formulation comprising ethanol, cromolyn sodium, optional surfactant and optional low volatility liquid from a liquid source to a flow passage;
(b) heating liquid aerosol formulation in a heated portion of the flow passage to produce a vapor; and
(c) admixing the vapor with air to produce an aerosol containing cromolyn sodium and optionally a surfactant and/or low volatility liquid such as propylene glycol.

10. The method of claim 9, wherein the liquid aerosol formulation further includes propylene glycol in an amount effective to prevent clogging of the flow passage.

11. A method of generating an aerosol, comprising:
(a) supplying a liquid aerosol formulation comprising a carrier, cromolyn sodium, optional surfactant and optional low volatility liquid to a flow passage;
(b) heating the liquid aerosol formulation in a heated portion of the flow passage to produce a vapor; and
(c) admixing the vapor with air to produce an aerosol.

12. A liquid aerosol suspension adapted to form a vaporization aerosol, comprising a high volatility carrier and a second component, the liquid aerosol formulation being propellant free and containing a zwitterionic and/or non-ionic surfactant.

13. A method of generating an aerosol, comprising:
(a) supplying a liquid aerosol suspension comprising a high volatility carrier and a second component to a flow passage and a zwitterionic and/or non-ionic surfactant;
(b) heating the liquid aerosol suspension in a heated portion of the flow passage to produce a vapor;
(c) admixing the vapor with air to produce an aerosol.

14. The method of claim 13, wherein the carrier comprises ethanol and the second component comprises cromolyn sodium.

15. The method of claim 13, wherein aerosol particles of the aerosol have a mass median aerodynamic diameter of about 0.5 to 5 microns.

16. The method of claim 13, wherein the liquid aerosol suspension further includes a low volatility liquid.

17. The method of claim 16, wherein the aerosol includes the surfactant and the low volatility liquid.

18. The method of claim 13, wherein the liquid aerosol suspension includes ethanol and propylene glycol, the heating vaporizes at least part of the ethanol and the vapor is jetted from the flow passage as a mist of aerosol particles containing non-vaporized liquid propylene glycol.

19. The method of claim 13, wherein the flow passage is a capillary sized flow passage.

20. A method of treating asthma in a subject in need thereof, comprising administering the aerosol produced according to the method of claim 13.
